(19) United States
(12) Patent Application Publication
(10) Pub. No.: US 2007/0196817 A1
(43) Pub. Date: Aug. 23, 2007

(54) Sampling Device

(75) Inventor: Murray Frederick Broom, Dunedin (NZ)

Correspondence Address: FOLEY AND LARDNER LLP;SUITE 500, 3000 K STREET NW, WASHINGTON, DC, 20007, US

(73) Assignee: Global Technologies (NZ) Ltd.

(21) Appl. No.: 10/568,705

(22) PCT Filed: Aug. 20, 2004
(86) PCT No.: PCT/NZ04/00191

§ 371 Date: Aug. 10, 2006

(30) Foreign Application Priority Data
Aug. 21, 2003 (NZ) NZ 527759

(51) Int. Cl. C12Q 1/70; C12Q 1/68; C12M 3/00; A61M 35/00

(52) U.S. Cl. 435/5; 435/6; 435/287.2; 604/1

(57) ABSTRACT

A method and a device for collecting a sample of material from a surface. A ball (2) within a socket (3) such that the ball is free to rotate in any direction. As the ball rolls across the surface the sample of material is collected by the ball and is transferred by the ball to a collection reservoir (not shown) via the conduit (14). An absorbent material may be placed in space (11) and a filter (10) in the outlet (9) is permeable to fluid but not absorbent material.

### FIELD OF INVENTION

This invention relates to a method and a device for collecting a sample of material from a surface. In particular, the invention relates to a device having a ball mounted within a socket, so that the ball is free to rotate in any direction, enabling the ball to be rolled across a surface. As the ball rolls across the surface, the sample of material is collected by the ball and is transferred by the ball to a collection reservoir. The sample may undergo analysis in the collection reservoir or may be transferred to another location in the device or, alternatively, to another device for analysis. The invention also includes a method of collecting a sample using the device.

### BACKGROUND

In many fields of technology samples of material, particularly samples of a fluid material or a material suspended in a fluid, must be extracted from a surface for subsequent analysis. There is a variety of well known devices available for achieving this, although many have disadvantages or have limited application to certain sampling and analysis processes.

Some known devices are instruments with sharp ends, such as biopsy or syringe needles. Needles often damage a surface when obtaining a fluid sample. In many instances, this is an undesirable but necessary consequence of using a needle. In addition, needles require a certain degree of care by the user to prevent injury to the user. Taking a sample of from a human or animal with a sharp-ended instrument can cause pain and injury.

Sharp-ended fluid samplers, such as needles, are also not desirable for use in some industries, such as the food production industry, due to the possibility of needles breaking or falling into the food and therefore posing a serious health and safety risk to consumers.

Another well known fluid sampling device is the pipette which is available in many different embodiments. Some pipettes are precision engineered instruments making them expensive and requiring expert knowledge for correct use. Other pipettes are simply a capillary with a rubber bung attached at one end to produce a vacuum when in use. This type of pipette does not yield reproducible sampling results and can not be adopted for high throughput automated sampling. Furthermore, neither pipettes nor needles are well suited to sampling very small volumes of fluid especially where the fluid is present as a very thin film.

Another device used to collect a sample of fluid material from a surface is a swab of absorbent material, such as a cotton bud. This type of device does not yield reproducible sampling results and has the added disadvantage that extracting the fluid for analysis after collection can be difficult. Furthermore, swabs are not suited to high throughput automated sampling.

More sophisticated devices based on the use of an absorbent material on a rotating element have been developed. For example, U.S. Pat. No. 5,554,537 describes a device having a flat element with a compressible absorbent material, surrounded by a chamber. In use, the wall of the chamber forms a seal against the surface from which a sample is to be taken. The absorbent material is then compressed between the sample surface and the flat element, and rotated in a direction parallel to the sample surface. This configuration has the advantage that the absorbent material can be pre-wetted and therefore a dry surface can be sampled. Furthermore, the rotation of the absorbent material effectively scrubs the surface thereby obtaining a better sample. However, correct operation of the device requires a seal to be formed between the walls of the device and the sample surface. This may be difficult or even impossible in some uses.

Another example is U.S. Pat. No. 6,266,838 which describes a device having a rotating drum with an absorbent material on its outer surface. The device is designed to mop up fluids spilt on a hard surface. The device also has an element that engages the absorbent surface and channels water away from the absorbent surface to a container as the drum rotates. This device suffers from the disadvantage that the absorbent material must be one that can be adhered to the drum, rather than a powder or granular resin as may be preferred in many uses. Further, this device is not well suited for sampling small volumes of fluid.

There is a need for a sampling device that can collect samples, including small sample volumes, from surfaces and can be operated without expert knowledge.

The applicant has now surprisingly found that a device having a ball in a socket is effective for collecting samples of material, where the device additionally has a means for transferring the sample to a reservoir in readiness for analysis of the sample.

Samples may be fluid samples, including suspensions in a fluid, or may be dry or almost dry solid samples that have the ability to adhere to the ball of the device.

Ball in socket devices are well known as devices for dispensing a fluid contained in a reservoir to a surface. Such devices include “roll-on” deodorants/antiperspirants, sunscreens, cosmetics, and other ointments or creams, as well as ballpoint writing pens.

A ball in socket dispensing device, however, does not have the same technical difficulties as a sampling device. A dispensing device needs a reservoir of fluid to be dispensing, but once dispensed the fluid requires no special treatment in relation to containment or application. In contrast, a sampling device must have an adequate means of collecting the sample from the ball and making it available for subsequent analysis. It is presumably for this reason that ball in socket sampling devices are unknown to date. The applicant has now discovered a way to successfully overcome these technical difficulties.

It is therefore an object of the invention to provide a sampling method and device that at least goes some way to overcoming the above disadvantages of known sampling methods and devices, or to at least provide a useful alternative.

### STATEMENTS OF INVENTION

In a first aspect of the invention there is provided a method of collecting a sample of material using a device having:
(a) a ball housed within a socket where at least part of the external surface of the ball is capable of contact with the sample, and
(b) a chamber shaped at one end to form the socket and at the other end to form a sample collection reservoir;
   where the ball maintained in contact with the material whilst the device is moved with respect to the material, such that the resulting rotation of the ball causes the sample of the material to be transferred to the collection reservoir.

Preferably the sample is a fluid or is suspended in a fluid. The method may include the transfer of the sample from the ball to the collection reservoir via an absorbent material housed within the collection reservoir and in contact the external surface of the ball.

The absorbent material may be any absorbent material suitable for the sample fluid, but, in the case of a fluid sample obtained for DNA analysis, is preferably a resin capable of deactivating nucleases, such as Chelex®. Alternatively, the absorbent material may be an absorbent membrane or filter. When the method is used for DNA collection and analysis, the absorbent membrane is preferably a metal chelating membrane.

Preferably the sample may also adhere to the external surface of the ball. The method may include the step of applying moisture or fluid to the material prior to collecting the sample.

The sample can be dissolved or suspended in a fluid in the collection reservoir.

Preferably the sample may also pass from the collection reservoir through an outlet in the device. The sample may pass into one or more conduits connected to the outlet.

In one embodiment the method includes the step of performing an analysis of the sample. The analysis can be performed in the collection reservoir, and may be performed while the sample is adhered to the external surface of the ball.

Alternatively, the analysis of the sample may be performed in a location in the device that receives the sample after it passes from the collection reservoir.

To perform the analysis the method can include the step of connecting the device to an analysis device for analysis of the sample. The external device may aid the analysis of the sample in the device, or it may receive the sample for analysis.

The analysis device may be a heating and/or cooling device, and can include a thermocycler.

To aid in the collection of the sample the ball can be chemically modified to enhance the affinity for the sample.

The method may be used to collect a biological, organic or inorganic sample. This may include, but is not limited to a biological cell, bacteria, a virus, a blood sample, a tissue sample, a plant sample, or an industrial waste sample.

The sample may be analysed using the present method for, but is not limited to, any one of DNA, RNA, an antigen, pathogens, a chemical contaminant, a trace element, or radioactivity.

In a second aspect of the invention there is provided a device for use in a method according to the first aspect of the invention.

The invention also provides for a device for collecting a fluid sample including:
(a) a ball housed within a socket where at least part of the external surface of the ball is capable of contact with the fluid,
(b) a chamber shaped at one end to form the socket and at the other end to form a sample collection reservoir,
(c) an absorbent material housed within the sample collection reservoir, where the external surface of the ball contacts the absorbent material.

Preferably the device includes an outlet to allow the fluid to pass from the reservoir. The device may further include one or more sample conduits connected to the outlet.

In one embodiment of the invention the device additionally includes an analysis means for analysing the sample. In an alternative embodiment, the device is adapted to be connected to an analysis device for analysis of the sample.

The device may be adapted so that analysis of the sample can occur when the sample is in the collection reservoir. Alternatively, the device may be adapted so that analysis of the sample occurs at a location in the device that receives the sample when it passes from the reservoir.

The device may also include a filter proximal to the outlet to contain the absorbent material in the reservoir but to allow sample to pass from the reservoir.

Preferably the device is longitudinal with a substantially circular external wall cross-section and houses the ball, the socket, and the chamber. The device preferably includes a handle formed as a shaft connected to the collection reservoir.

The handle may be integrally formed with the socket or, alternatively, the socket may be mounted to the handle so that the handle is detachable.

The device preferably includes a cap for each end of the device so that the device can be sealed both before and after collecting a sample.

The surface of the ball may be smooth, or may be textured or roughened to minimise slippage of the ball on a surface when in use. The surface of the ball may also be chemically modified to enhance the affinity for the sample.

The device preferably includes a temperature control means to heat or cool the sample once collected. Preferably the temperature control means is a heating element located in the socket or in the handle of the device.

The device may be constructed of any suitable material, but preferably a metal or a plastics material.

### BRIEF DESCRIPTION OF FIGURES

FIG. 1 shows a cross sectional representation of a device according to the present invention

FIG. 2 shows the uptake of a fluid using the method and a device according to the present invention.

FIG. 3 shows the detection of adenylate kinase from a tissue sample using the method and a device according to the present invention

FIG. 4 shows the detection of bacteria in a sample using the method and a device according to the present invention

### DETAILED DESCRIPTION

The method and device of the invention is intended to be used to obtain samples of material from a surface. The material may be biological, such as blood, or non-biological, such as waste from a chemical plant, and may be synthetic or non-synthetic. The material may be a fluid material, including any material or substance suspended or dissolved in a fluid. The material may also be a solid or semi-solid material, including a particulate or microparticulate material.

In use, the ball of the device is rolled across the surface on which the sample to be collected and analysed is present. As the ball rotates, the sample is transferred to the sample collection reservoir.

A liquid carrier may be used to transfer the sample from the sample collection reservoir to a location in the device for analysis of the sample or to an analysis device connected to the sample collection reservoir.

Alternatively, the sample may be contacted with an absorbent material in the sample collection reservoir as the ball with sample on its external surface rotates. The ball is therefore in direct contact with the absorbent material to ensure that the fluid sample contacts the absorbent material and is retained in the reservoir. This method of sample transfer is most suited to a fluid sample.

The term “absorbent material” is intended to cover any absorbent substance required for the sampling being undertaken, whether particulate, granular, powdery, fibrous or a solid porous matrix such as a sponge, whether synthetic or non-synthetic, whether hydrophobic or hydrophilic and whether inert or reactive with the sample fluid.

The device of the invention has the particular advantages that it is easy to use, expert knowledge is not required to operate the device, and the device can be easily manufactured. Further, the sampling method is non-invasive and painless. Additionally, a ball and socket sampling device does not pose the same risk to users as with sharp sampling devices such as needles.

Once the sample has been obtained a cap can be placed over the exposed ball allowing indefinite storage of the sample. Alternatively, the sample can be analysed. The analysis can occur directly in the collection reservoir, and even while the sample is still adhered to the ball surface. For example, the collection reservoir may contain, or have added to, reagents to detect the presence or absence of a compound or substance. Alternatively, the sample can be transferred to another location within the device, or to another analysis device for analysis. This may be achieved by dissolving or suspending the sample in a fluid in the collection reservoir.

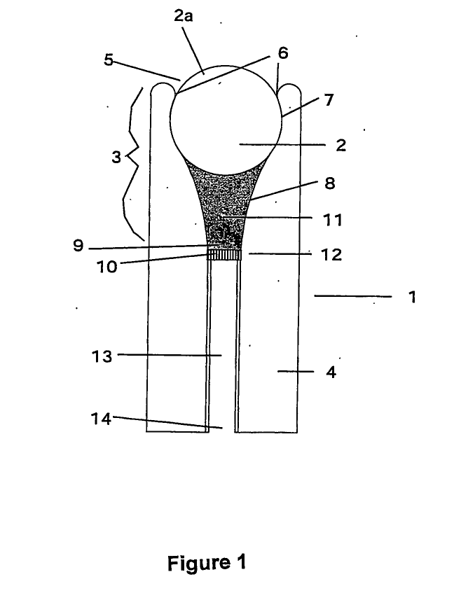

The device used in the method is further described by way of example with reference to FIG. 1. It is to be appreciated that the device shown in FIG. 1 is one example of the invention and that the invention is not limited to the example.

A sampling device 1 comprises a solid sampling ball 2 housed within a socket 3. The ball 2 is free to rotate in any direction and has a surface that can range from substantially smooth to heavily textured. The socket 3 is attached to or built into the end of a handle 4. The handle of a device of the invention may be configured to suit a particular application for the sampling device. In the device 1 illustrated in FIG. 1 the handle 4 is a shaft.

The socket 3 comprises an opening 5, through which the ball 2 is exposed and is accessible to a wetted surface for sampling. The small gap between the ball 2 and the socket 3 represents the sample inlet 6. The diameter of the socket opening 5 is smaller than the diameter of the ball 2, thus preventing the ball 2 from falling out of the socket 3.

The inner wall of the socket 3 has a concave portion 7 so that there is a snug fit with the ball 2. The inner wall also has a tapered portion 8 that tapers away from the ball 2 to form a space 11 behind the ball 2. The space 11 has a sample outlet 9, which is sealed with a physical barrier 10. The sample outlet 9 is shown in longitudinal alignment in the device 1 with the socket opening 5. However, it is to be appreciated that the sample outlet 9 may be positioned at any angle relative to the socket opening 5.

The socket 3 is shown integrally formed with the handle 4 of the sampling device 1. However, the socket 3 may be independently mounted to the handle 4 in an alternative construction of the device.

The space 11 may contain an absorbent material which is in direct physical contact with the ball 2. This contact is necessary to enable fluid to be absorbed into the absorbent material. As will be appreciated by those skilled in the art, the absorbent material will be selected depending upon the nature of the fluid being sampled and/or the type of subsequent analysis to be carried out on the sample. The nature of the absorbent material needs to be consistent with the wetting fluid or fluid to be sampled. For example, a sample of hydrophobic fluid will require that a hydrophobic absorbent material is used in the sampling device.

The absorbent material is held in place within the device 1 by the ball 2 and the barrier 10. The ball 2 is effectively a valve retaining the absorbent material within the device 1 yet allowing the passage of air and sample during the rolling of the ball. The gap 6 between the socket wall 7 and the ball 2 is sufficiently small that the absorbent material in the space 11 cannot leak out of the socket opening 5. The barrier 10 at the sample outlet 9 must be permeable to air, wetting fluid and sample fluid to enable fluid to move through the absorbent material. However, the barrier 10 must not be permeable to the absorbent material. The barrier 10 is therefore a filter.

The sample fluid outlet 12, comprising the outlet 9 and barrier 10, is connected to a conduit 13 through which the sample fluid can be transported by applying a vacuum to the conduit outlet 14. The conduit 13 is housed within the handle 4 of the sampling device 1.

Both ends of the sampling device 1 can be sealed before and after use with removable caps (not shown).

The handle 4 of the sampling device 1, and the conduit 13 housed within, may be configured with adaptors or fittings to facilitate connection to other devices for further processing or analysis of the sample fluid.

The sampling ball 2, socket 3, and the handle 4 may be constructed with a suitable metal, or a plastics material, or composite materials.

In operation, the ball 2 extracts a sample from a surface by making physical contact with the surface. The ball 2 is rolled over the surface in one or more back and forth strokes, or circular movements.

The surface being sampled is preferably sufficiently textured to provide enough purchase to prevent slippage of the ball 2 and to enable the ball 2 to rotate within the socket 3. In situations where the sample surface is too smooth, a portion of the sample can be put onto an artificial textured sampling surface such as a gauze mat.

The surface to be sampled may be inherently wet or it may be dry. If the surface is dry either the surface or the sampling device may be subjected to an extra preparative step prior to the sampling process. A dry surface can be wetted prior to sampling by applying a wetting fluid, or a fluid containing a substance or material to be sampled, using any wetting method but preferably spraying. Alternatively, a dry surface can be sampled using a pre-wetted sampling device 1. When the device 1 is pre-wetted, the absorbent material in the space 11 is fully or partially saturated with wetting fluid.

Particulate or microparticulate material from a dry surface can be directly sampled. In this application the device is rolled over a substantially dry surface. Material adheres to the surface of the ball and is transferred into the collection reservoir within the device.

The ball in socket confers a number of mechanical advantages for sampling. For instance, in some applications mechanical disruption of a sample may be advantageous (e.g. cell rupture for DNA analysis). The ball in socket device can facilitate mechanical disruption of tissue by mechanically crushing the tissue as it rolls over the sample and by breaking cells away from the tissue mass via the shearing force of the ball 2 rotating within the socket 3 causing tissue teasing, cell dispersal, and some fragmentation of cells. The sample inlet 6 represents a capture zone for facilitating the transfer of fluid and material into the device as the ball rolls. The close tolerance of the ball within the socket defines and limits the particle sizes that can enter the device.

In some applications it is desirable that the sample fluid, during or after extraction from the wetted surface, is heated and/or cooled. This can be achieved by placing either the entire device 1, or the exposed portion 2a of the ball 2 into or onto a suitably configured temperature controlled device. Physical contact between the device 1 and the temperature controlled device facilitates heat transfer between the said devices, resulting in heating/cooling of the sampling device. Alternatively, the device 1 can be configured with a built-in means of heating, such as a heating element located in the socket 3 and/or handle 4.

When a heating or cooling step is desirable, either the ball 2 or the device 1 is preferably constructed of a fully- or semi-heat conducting material. However, small scale devices manufactured from non-conducting plastics will also rapidly equilibrate to the temperature of the temperature controlled device. It will be appreciated that both the temperature and the period of heating will be selected depending upon the nature of the sample and/or the nature of the absorbent material and/or the type of subsequent analysis to be carried out on the sample.

If there is significant evaporation of water from the absorbent material during heating of the device 1, water can be replaced by rolling the ball 2 over a rehydration fluid, which will be wicked up and hydrate the absorbent material.

After a surface has been sampled, and a sample has been transferred to the collection reservoir, the sample can be directly removed from the device 1 or it can be stored within the device 1 for a period of time. The preferred method of removing a sample from the device 1 is by applying a vacuum to the conduit outlet 14. This can be done either directly by connecting a vacuum device, e.g. a syringe, directly to the conduit outlet 14, or indirectly by applying a vacuum to other conduits or chambers that may be connected (not shown) to the conduit outlet 14.

The invention can be operated manually, or alternatively can be robotically controlled and operated throughout the sampling process.

One possible application of the invention is in the sampling of biological samples (e.g. meat, skin, plant material, microbial cultures) for the purpose of extracting cells and optionally for processing the cells in readiness for subsequent analysis, such as DNA, protein, carbohydrate or lipid analysis. If the device 1 is used to obtain a sample for DNA analysis, the absorbent material is preferably an absorbent resin covalently linked with a chelator of bivalent cations, e.g. Chelex®, which, by chelating bivalent cations, leads to the inactivation of nucleases.

During operation of the device 1 for obtaining DNA analysis samples, the ball 2 picks up or extracts the sample fluid from a surface. In some applications, the sample may then be transferred onto an absorbent surface within the device. The sample may contain sufficient moisture to facilitate this. Alternatively, the device may contain a pre-packaged fluid to aid transfer of the sample from the ball surface to the absorbent material (e.g. Chelex® or a chelating membrane).

The device 1 is preferably heated to facilitate heating of the resin to a temperature in the range 75° C. to 98° C. The heating protocol is selected according to the heat conductance of the device 1 or the ball 2, and according to what temperature is reached in the absorbent material. For instance, the device can be heated for 4 minutes if the resin temperature is 750C. The purpose of heating the device 1 in this operation is to promote lysis of the cells in the sample fluid, inactivate nucleases released from the cells, and denature the protein scaffold in chromatin. The DNA from the cells is therefore stably prepared and accessible for subsequent processing or analysis.

The device of the invention may be used in any application where high throughput, automated sampling is desirable. The device can be constructed as a portable pocket-sized device for low throughput manual sampling. The device will in most applications be used only once and will be disposed of after a sample has been collected and processed.

The device of the invention has the particular advantage that it is very easy to use and does not require expert knowledge to operate.

A further advantage of the invention is that the device can be configured to sample small volumes of fluid (in the millilitre range or microlitre range) by changing the volume of absorbent material in the device.

Other advantages include:
ability to obtain small sample volumes
non-invasiveness
avoidance of hazards associated with using sharp needles
minimal disruption or damage to surface from which sample is obtained
suitability for high throughput sampling
portability
robust design
easily and cheaply manufactured

The invention is further described with reference to the following examples. It is to be appreciated that the invention is not limited in any respect by the examples.

### EXAMPLES

### Example 1

### Transfer of Fluids into an Absorbent Reservoir

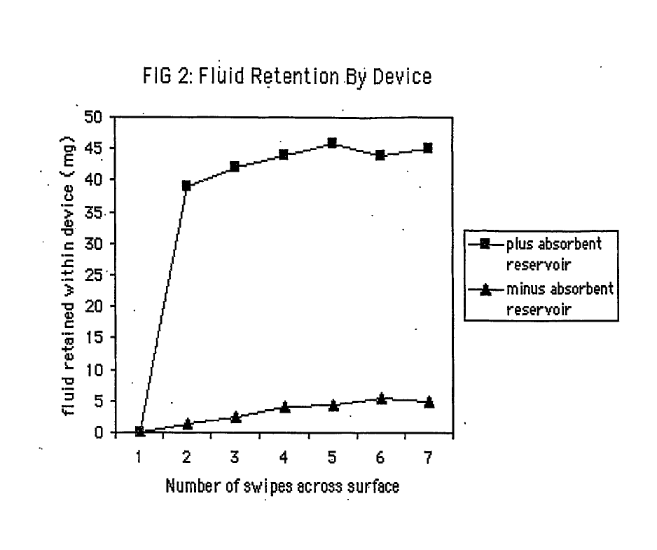

A series of experiments were performed using sampling devices of the invention similar to the device shown in FIG. 1. The barrier 10 consisted of a porous glass filter, the absorbent material 11 consisted of a hydrophilic resin, the ball and the holder were polypropylene. The sampling surface consisted of water soaked gauze in a small petri dish. The water was coloured with food colouring. The ball was rolled across the surface 1-2 times and the fluid was observed to rapidly saturate the absorbent resin 11. Verification of the uptake was visual because of the translucent nature of the device and the coloured fluid. The devices were also weighed before and after the experiment. The fluid uptake was shown to be proportional to the volume of the absorbent resin. The device packed with 100 mg of a hydrophilic resin would absorb approximately 45 mg of fluid. Without the absorbent resin, the device would absorb approximately 6 mg of fluid (see below FIG. 2).

### Example 2

### Transfer of Sample onto the Ball Surface

A series of experiments were performed using sampling devices of the invention, similar to the device shown in FIG. 1 with the following modifications. The device lacked the absorbent resin and the glass filter and was sectioned behind the ball so that access was provided to the internal face of the ball.

(a) Meat Biopsy

The device was rolled across damp gauze that contained a meat tissue biopsy. The rolling caused the crushing of the tissue. Sample was transferred into the device through rolling across the gauze surface. The internal face of the ball was sampled enzymatically for biological activity using adenylate kinase as the marker enzyme. The inner ball surface was touched with 20 μl fluid. This fluid was then removed and analysed for the presence of adenylate kinase using luciferase bioluminescence. The control experiment consisted of rolling the device across damp gauze without the meat biopsy and tested the internal ball surface for enzyme activity. The total volume of the assay was 0.2 ml. The assay conditions were 0.18 mM glycylglycine ph 8.0, MgCl₂ 10 mM, adenosine diphosphate 250 μmolar, luciferin 33 μmolar, luciferase 5 μg, coenzyme A 1 mM. The bioluminescence was determined using a photomultiplier tube and the photon-counting package from Electron Tubes Limited.

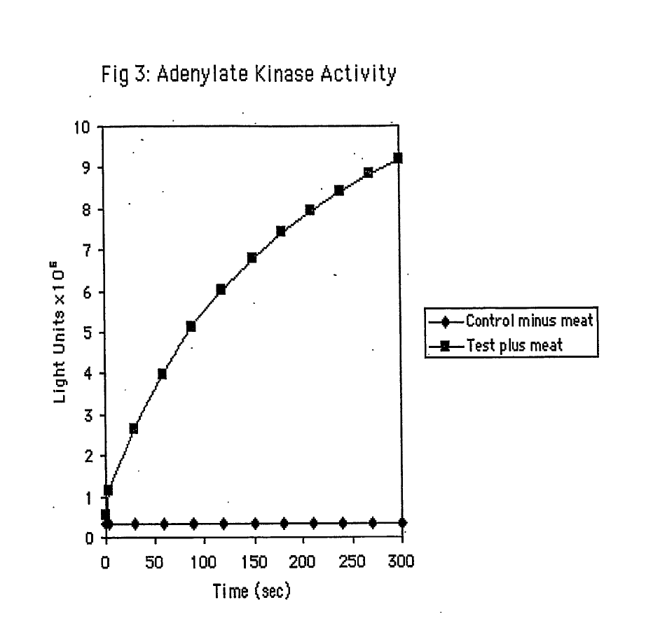

The results in FIG. 3 show that adenylate kinase could be detected on the surface of the ball within the collection reservoir, while no adenylate kinase was present on the control. The experiment shows that the device can be used to collect trace amounts of a sample on the ball surface sufficient for further analysis.

(b) Bacterial Samples

The ball device was rolled across a gauze surface that contained E. coli (3.6×10⁷ cells). The internal ball surface was sampled as with the meat biopsy experiment. A 20 μl sample was touched on the interior ball surface and removed to determine biological activity. In these assays, the fluid sample contained 6.25 mM Tris buffer (pH 8.0), 12.5 mm glucose, 0.1% Triton X-100 and 2 mg lysozyme. After 10 minutes pre-incubation at room temperature, the sample was added to a cuvette containing the reagents for adenylate kinase determination and bioluminescence was determined, again as above for the meat biopsy experiment. The control sample measured adenylate kinase activity derived from rolling the ball across a damp gauze surface in the absence of E. coli.

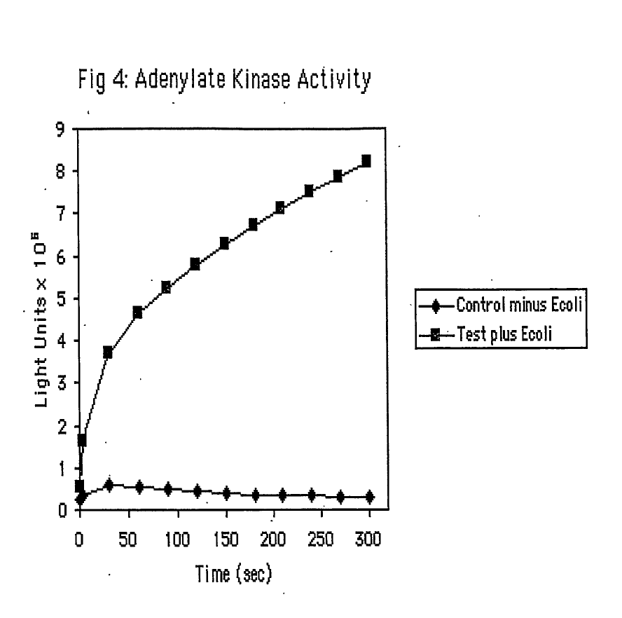

The results in FIG. 4 show that adenylate kinase activity from the bacteria could be detected on the ball surface. No adenylate kinase activity could be detected on the control device. This experiment further illustrates that the present method and device can be used to obtain a sample of material on the ball surface which when retained in the collection reservoir can be subjected to an analysis or stored. For example, as illustrated in the present example the method and device can be used to detect bacterial contamination on a surface. The surface could range from a food surface to a food preparation surface or a hospital surface.

Although the invention has been described by way of example, it should be appreciated that variations and modifications may be made without departing from the scope of the invention as claimed. Furthermore, where known equivalents exist to specific features, such equivalents are incorporated as if specifically referred in this specification.

### INDUSTRIAL APPLICABILITY

The device of the invention may be used in a wide variety of industrial applications. These include the testing of foods, human or animal fluid and tissue testing, environmental waste and hazardous substance testing

The method and device of the present invention may be used at a wide variety of industrial applications. This includes, but is not limited to, the testing of foods, human or animal fluid and tissue testing, environmental waste and hazardous substance testing. Applications in food processing facilities may include the use of the ball to collect samples of surface bacteria to screen for the presence of pathogens, such as listeria or salmonella. Similarly the device could be used in food assurance applications. For example, the method and device may be used to collect samples from beef to screen for the presence of BSE. More generally the method and device may be used to collect samples for the purpose of conducting DNA analyses. These analyses may be for medical diagnostics, organism detect or individual identification. Other industrial applications include collecting samples to test for the presence or absence of particular chemicals or organisms. This may, for example, be a part of quality control. In the case of hazardous substance testing this might involve the identification of poisons in industrial effluent through to screening for bio-terror elements such as anthrax.

1-42. (canceled)

43. A method of collecting a sample from a material comprising:
(a) providing a device comprising:
(i) a chamber shaped at one end to form a socket and at the other end to form a sample collection reservoir; and
(ii) a ball housed within the socket, wherein at least part of an external surface of the ball in the socket is configured to contact the sample;
(b) introducing the material to the device;
(c) rotating the ball; and
(d) collecting the sample in the collection reservoir.

44. The method as claimed in claim 43 wherein the material is transferred from the ball to the collection reservoir via an absorbent material housed within the collection reservoir and in contact with the external surface of the ball.

45. The method as claimed in claim 44 wherein the absorbent material is selected from the group consisting of a resin capable of deactivating nucleases, an absorbent membrane, absorbent filter, and a metal chelating membrane.

46. The method as claimed in claim 43 further comprising applying moisture or fluid to the material prior to collecting the sample.

47. The method as claimed in claim 43 further comprising dissolving or suspending the sample in a fluid in the collection reservoir.

48. The method as claimed in claim 43 wherein the sample passes from the collection reservoir through an outlet.

49. The method as claimed in claim 48 wherein the sample passes into one or more conduits connected to the outlet.

50. The method as claimed in claim 43 further comprising performing an analysis of the sample.

51. The method as claimed in claim 50 wherein the analysis is performed in the collection reservoir.

52. The method as claimed in claim 50 further comprising connecting the device to an analysis device for analysis of the sample.

53. The method as claimed in claim 52 wherein the analysis device is a thermocycler.

54. The method as claimed in claim 43 wherein the sample is selected from the group consisting of a biological cell, a bacterium, a virus, a blood sample, a tissue sample, a plant sample, and an industrial waste sample.

55. The method as claimed in claim 50 wherein the analysis comprises evaluating a substance selected from the group consisting of DNA, RNA, an antigen, a pathogen, a chemical contaminant, a trace element, and radioactivity.

56. A device for collecting a fluid sample including:
(a) a ball housed within a socket where at least part of the external surface of the ball is capable of contact with the fluid,
(b) a chamber shaped at one end to form the socket and at the other end to form a sample collection reservoir,
(c) an absorbent material housed within the sample collection reservoir, where the external surface of the ball contacts the absorbent material.

57. A device as claimed in claim 56 further comprising an outlet to allow fluid to pass from the reservoir.

58. A device as claimed in claim 57 further comprising one or more sample conduits connected to the outlet.

59. A device as claimed in claim 56 wherein the device is adapted to connect to an analysis device for analysis of the sample.

60. A device as claimed in claim 56 wherein the device is adapted so that analysis of the sample can occur when the sample is in the collection reservoir.

61. A device as claimed in claim 56 wherein the device is adapted so that analysis of the sample occurs at a location in the device that receives the sample when it passes from the reservoir.

62. A device as claimed in claim 56 wherein the absorbent material is selected from the group consisting of a resin capable of deactivating nucleases, an absorbent membrane, absorbent filter, and a metal chelating membrane.

63. A device as claimed in claim 56 wherein the device is longitudinal with a substantially circular external wall cross-section and houses the ball, the socket, and the chamber.

64. A device as claimed in claim 56 wherein the device comprises a handle formed as a shaft connected to the collection reservoir.

65. A device as claimed in claim 64 wherein the handle is integrally formed with the socket.

66. A device as claimed in claim 64 wherein the socket may be mounted to the handle so that the handle is detachable.

67. A device as claimed in claim 56 comprising a cap for each end of the device so that the device can be sealed both before and after collecting a sample.

68. A device as claimed in claim 56 wherein the surface of the ball is textured or roughened to minimize slippage of the ball on a surface when in use.
